(19) United States
(12) Patent Application Publication
(10) Pub. No.: US 2005/0249786 A1
(43) Pub. Date: Nov. 10, 2005

(54) Hydrophilic dispersions of nanoparticles of inclusion complexes of amorphous compounds

(75) Inventors: Rina Goldshtein, Har Hebron (IL); Irene Jaffe, Yavne (IL); Boris Tulbovich, Ashkelon (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 11/100,609

(22) Filed: Apr. 7, 2005

Related U.S. Application Data
(63) Continuation-in-part of application No. 10/952,380, filed on Sep. 29, 2004.
(63) Continuation-in-part of application No. 10/256,023, filed on Sep. 26, 2002.
Application No. 10/256,023 is a continuation-in-part of application No. 09/966,847, filed on Sep. 28, 2001, now Pat. No. 6,878,693.
(60) Provisional application No. 60/507,623, filed on Sep. 30, 2003.

(51) Int. Cl. A61K 31/7048; A23K 1/165; A61K 9/48; A61K 9/14

(52) U.S. Cl. 424/442; 514/28; 514/29; 424/489

(57) ABSTRACT

The present invention provides a hydrophilic inclusion complex consisting essentially of nanosized particles of an active compound in amorphous form and an amphiphilic polymer which wraps the active compound such that non-valent bonds are formed between the active compound and the amphiphilic polymer. The invention further provides hydrophilic dispersions comprising said inclusion complexes, particularly of pharmaceutical drugs, and stable pharmaceutical compositions comprising said dispersions of the pharmaceutical drugs in amorphous form.

### CROSS-REFERENCE TO RELATED APPLICATIONS

The present application is a continuation-in-part of application Ser. No. No. 10/952,380, filed Sep. 29, 2004, which is a non-provisional of the Provisional Application No. 60/507,623, filed Sep. 30, 2003 and which is a continuation-in-part of application Ser. No. 10/256,023, filed Sep. 26, 2002, which is a continuation-in-part of application Ser. No. 09/966,847, filed Sep. 28, 2001, the entire contents of each and all these applications being hereby incorporated by reference herein in their entirety as if fully disclosed herein.

### FIELD OF THE INVENTION

The present invention is in the field of nanoparticles. More particularly, the invention relates to soluble nanosized particles consisting of inclusion complexes of active amorphous compounds surrounded by and entrapped within suitable amphiphilic polymers, and to methods of producing such soluble nanoparticles.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions.

The size of the drug molecules also plays a major role in their solubility and stability as well as bioavailability. Bioavailability refers to the degree to which a drug becomes available to the target tissue or any alternative in vivo target (ie., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Liposomes, as drug carriers, have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity. The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability.

For a long time, most cyclodextrins had been no more than scientific curiosities due to their limited availability and high price, but lately cyclodextrins and their chemically modified derivatives became available commercially, generating a new technology of packing on the molecular level. Cyclodextrins are, however, fraught with disadvantages including limited space available for the active molecule to be entrapped inside the core, lack of pure stability of the complex, limited availability in the marketplace, and high price.

Microencapsulation is a process by which tiny parcels of a gas, liquid, or solid active ingredient (“core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include the use of toxic emulgators in emulsions or dispersions, polymerization or the application of high shear forces during emulsification process, insufficient biocompatibility and biodegradability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer or “generation” to the growing polymer. Dendrimers of up to ten generations have been synthesized with molecular weights in excess of 106 kDa. One important feature of dendrimeric polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as an efficient drug delivery system.

In the pharmaceutical industry, it is important to secure the stability and effectiveness of the products. The crystalline state of the active ingredient in a solid pharmaceutical preparation is known to affect physicochemical stability, solubility and absorption of a pharmaceutical drug and, thus, play a significant role in the behavior of the drug and may influence its therapeutical effect.

With the recent increase in the speed of development of new drugs and biotechnologies, determining the crystallinity of an organic material has become increasingly important. A number of methods have been developed for this purpose, including X-ray diffraction (XRD), a method unique in its ability to study the microstructure of materials. The degree of crystallinity affects not only the long-term stability of a pharmaceutical, but also its biological activity, which can mean the difference between toxic doses and ineffective doses. Clearly, potentially toxic or unstable drug formulations are to be avoided at all costs, making crystallinity determination a critical analysis for the pharmaceutical industry.

The amorphous state is characterized by a disordered molecular or atomic arrangement. Pharmaceutical drugs in the amorphous state are more soluble than the crystalline form and have increased bioavailability. However, due to the instability of many amorphous formulations, the pharmaceutical industry has not yet embraced these formulations and most pharmaceutical drugs are derived from crystalline active compounds.

Drugs can be produced in amorphous form by several methods including spray drying and grinding. The use of spray drying is known is disclosed, for example, in U.S. Pat. No. 6,763,607, EP 0901786, EP 1027886, EP 1027887, EP 1027888, WO 00/168092 and WO 00/168055. The preparation of amorphous forms of the macrolide antibiotic clarithromycin by grinding and spray drying gave products showing tendence toward crystallization, although with increased grinding time the amorphous state tending to resist crystallization was formed (Yonemochi E. et al., 1999 Eur. J. Pharm. Sci. 7:331-338).

Donepezil, 1-benzyl-4-((5,6-dimethoxy-1-indanon)-2-y1)methylpiperidine, and analogues, were described in U.S. Pat. No. 4,895,841 as acetylcholinesterase inhibitors and useful for treatment of various kinds of dementia including Alzheimer senile dementia, Huntington's chorea, Pick's disease, and ataxia. Donepezil hydrochloride is a white crystalline powder and is freely soluble in chloroform, soluble in water and in glacial acetic acid, slightly soluble in ethanol and in acetonitrile and practically insoluble in ethyl acetate and in n-hexane. Donepezil hydrochloride is available for oral administration in film-coated tablets containing 5 or 10 mg of donepezil hydrochloride for treatment of mild to moderate dementia of the Alzheimer's type. U.S. Pat. No. 5,985,864 and U.S. Pat. No. 6,140,321 disclose donepezil in the form of four polymorphs which are stable against heat and humidity. Recently, U.S. Pat. No. 6,734,195 disclosed that wet granulation of donepezil hydrochloride yields, after drying and milling, a stable granulate that uniformly contains donepezil hydrochloride amorphous.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention applies the technology of solumerization disclosed in the above-mentioned parent U.S. application Ser. No. 10/952,380, Ser. No. 10/256,023, and Ser. No. 09/966,847, incorporated herewith by reference in their entirety, for the preparation of nanodispersions of active compounds in amorphous form.

Thus, the present invention relates to a hydrophilic inclusion complex consisting essentially of nanosized particles of an active compound in amorphous form and an amphiphilic polymer which wraps the active compound such that non-valent bonds are formed between the active compound and the amphiphilic polymer in said inclusion complex.

The present invention further relates to hydrophilic dispersions comprising nanoparticles of said inclusion complexes, to their preparation and to stable pharmaceutical compositions comprising said dispersions.

### BRIEF DESCRIPTIONS OF THE DRAWINGS

FIG. 1 illustrates the X-ray diffraction pattern of powder crystalline donepezil hydrochloride (DH) (curve 1) and of the inclusion complex of DH-hydrolyzed potato starch (HPS) (curve 2).

FIG. 2 illustrates differential scanning calorimetry (DSC) analysis of commercially available donepezil hydrochloride powder.

FIG. 3 illustrates DSC analysis of donepezil hydrochloride-HPS inclusion complex sample of FIG. 1.

FIG. 4 illustrates an electron micrograph of nanoparticles of donepezil hydrochloride -HPS inclusion complexes having a size of approximately 100 nm.

FIG. 5 illustrates the size distribution of nanoparticles comprising donepezil-modified starch inclusion complexes (#LG-7-51, Table 1) having a size of approximately 600 nm, as measured by light diffraction (ALV).

FIG. 6 illustrates the X-ray diffraction pattern of alginate (curve 3) compared to donepezil hydrochloride-alginate inclusion complex samples (curve 1 and 2). Sample 1 (curve 1) was prepared without adding methyl acetate to the aqueous alginate solution along with adding the active compound dissolved in dichloromethane, and sample 2 (curve 2) was prepared with the addition of methyl acetate.

FIG. 7 illustrates the size distribution of nanoparticles of itraconazole-modified starch inclusion complexes having a size of approximately 100 nm, as measured by light diffraction (ALV).

FIGS. 8A-8B illustrate X-ray diffraction patterns of conmmercially available itraconazole (7A) and of itraconazole-acrylate copolymer inclusion complex (7B).

FIG. 9 illustrates DSC analysis of commercially available itraconazole.

FIG. 10 illustrates DSC analysis of itraconazole-acrylate copolymer inclusion complex sample of FIG. 8B.

FIGS. 11A-11B illustrates DSC analysis of commercial crystalline itraconazole (11A) and of nanoparticles comprising itraconazole-polyacrylic acid inclusion complexes (#IT-56, Table 2).

FIG. 12 illustrates X-ray diffraction pattern of 2-month old azithromycin-HPS inclusion complex sample (curve 2) compared to the commercially available azithromycin (curve 1).

FIG. 13 illustrates DSC analysis of commercially available azithromycin.

FIG. 14 illustrates DSC analysis of azythromycin (2%)-alginate inclusion complex sample of FIG. 15.

FIG. 15 illustrates X-ray diffraction pattern of azythromycin (1%)-alginate inclusion complex sample (curve 1) compared to of azythromycin (2%)-alginate inclusion complex sample (curve 2); both samples were 6-month old.

FIG. 16 illustrates the size distribution of nanoparticles comprising azithromycin-chitosan inclusion complexes (#10-148/2, Table 3) having a size of approximately 362 nm, as measured by light diffraction (ALV).

FIG. 17 illustrates X-ray spectra of 10-month old azithromycin-chitosan inclusion complex sample (bottom curve) compared to the commercially available azithromycin (upper curve).

### DETAILED DESCRIPTION OF THE INVENTION

The present invention provides nanoparticles and methods for the production of soluble nanoparticles and, in particular, hydrophilic dispersions of nanoparticles of inclusion complexes of an active compound in amorphous form enveloped in amphiphilic polymers.

The soluble nanoparticles, referred to sometimes herein as “solunanoparticles” or “solumers”, are differentiated by the use of water-soluble amphiphilic polymers that are capable of producing molecular complexes with active molecules, particularly pharmaceutical drugs. The solunanoparticles formed in accordance with the present invention render water-insoluble active compounds soluble in water and readily bioavailable in the human body.

As used herein, the term “inclusion complex” refers to a complex in which one component—the amphiphilic polymer (the “host), forms a cavity in which molecular entities of a second chemical species—the active compound (the “guest”), are located. Thus, in accordance with the present invention, inclusion complexes are provided in which the host is the amphiphilic polymer and the guest is the active molecule in amorphous form wrapped and fixated or secured within the cavity or space formed by said amphiphilic polymer host.

In accordance with the present invention, the inclusion complexes contain the active compound in amorphous form, which interacts with the polymer by non-valent interactions and form a polymer-active as a distinct molecular entity. A significant advantage and unique feature of the inclusion complex of the present invention is that no new chemical bonds are formed and no existing bonds are destroyed during the formation of the inclusion complex (very important for pharmaceutical drugs). The particles comprising the inclusion complexes are nanosized and no change occurs in the active compound molecule itself, when it is enveloped, or advantageously wrapped, by the polymer.

Another important characteristic of the inclusion complex of the invention is that the active compound is in the amorphous state. It is known in the art that the amorphous state is preferred for drug delivery as it may indeed enhance bioavailability.

The creation of the complex does not involve the formation of any valent bonds (which may change the characteristics or properties of the active compound). As used herein, the term “non-valent” is intended to refer to non-covalent, non-ionic and non-semi-polar bonds and/or interactions, and includes weak, non-covalent bonds and/or interactions such as electrostatic forces, Van der Waals forces, and hydrogen bonds formed during the creation of the inclusion complex. The formation of non-valent bonds preserves the structure and properties of the active compound.

The solunanoparticles of the invention remain stable for long periods of time, may be manufactured at a low cost, and may improve the overall bioavailability of the active compound.

In one aspect, the present invention relates to a hydrophilic inclusion complex consisting essentially of nanosized particles of an active compound in amorphous form and an amphiphilic polymer which wraps the active compound such that non-valent bonds are formed between the active compound and the amphiphilic polymer.

The amphiphilic polymer is preferably selected from the group of biocompatible polymers, more preferably those approved for human use. Such polymers comprise, for example, but are not limited to, natural polysaccharides, modified polysaccharides, polyacrylic acid and copolymers thereof, polymethacrylic acid and copolymers thereof, polyacrylamide and copolymers thereof, polymethacrylamide and copolymers thereof, polyethylene imine, polyethylene oxide, polyvinyl alcohol, polyisoprene, polybutadiene, and gelatin.

In one embodiment, the amphiphilic polymer is a polysaccharide selected from the group consisting of natural or modified starch, chitosan and alginate.

In one embodiment, the polysaccharide is starch that should preferably have a large proportion of linear chains, i.e. starch with high contents of amylose, the constituent of starch in which anhydroglucose units are linked by D-1,4 glucosidic bonds to form linear chains, and low contents of amylopectin, a constituent of starch having a polymeric, branched structure. The levels of amylose and amylopectin and their molecular weight vary between different starch types.

To improve its characteristics for use in the invention, starch, e.g. corn or potato starch, can be modified, for example by increasing its hydrophilicity by acid hydrolysis, e.g., with citric acid, and/or by reaction with an agent, e.g. polyethylene glycol (PEG) and/or hydrogen peroxide. In addition, starch can be subjected to thermal treatment, for example at 160-180° C., for about 30-60 min, to reduce the amount of branching.

The active compound is any active compound that is desired to be obtained in amorphous form. It may be a water-insoluble or a partially or fully water-soluble compound, for example, as described in the above-mentioned parent U.S. application Ser. No. 10/952,380, Ser. No. 10/256,023, and Ser. No. 09/966,847, incorporated herewith by reference in their entirety. The active compound may be selected from the group consisting of pharmaceutical compounds, food additives, cosmetics, pesticides and pet foods.

The active compound is preferably a pharmaceutical compound, but also compounds for agricultural use, e.g. pesticides, cosmetic and food additive uses are encompassed by the present invention. The active compound can be small or large, simple or complex, heavy or light and include macromolecular compounds such as polypeptides, proteins, nucleic acids and polysaccharides.

In one embodiment, the active compound in amorphous form is a macrolide antibiotic selected from the group consisting of erythromycin, clarithromycin and azithromycin.

In one embodiment, the present invention provides a hydrophilic inclusion complex consisting essentially of nanosized particles of amorphous azithromycin wrapped in a polysaccharide selected from the group consisting of natural or modified starch such as hydrolyzed potato starch (HPS), chitosan or alginate.

In another embodiment, the present invention provides a hydrophilic inclusion complex consisting essentially of nanosized particles of amorphous clarithromycin wrapped in a polysaccharide selected from the group consisting of starch such as hydrolyzed potato starch (HPS), chitosan or alginate.

In another embodiment, the amorphous active compound is an azole compound. In accordance with the present invention, an “azole compound” refers to imidazole and triazole compounds for human or veterinary application or for use in the agriculture.

In one preferred embodiment, the azole compound is selected from azole fungicides for human application used in many different antimycotic formulations including, but not limited to the triazoles terconazole, itraconazole, and fluconazole, and the imidazoles clotrimazole, miconazole, econazole, ketoconazole, tioconazole, isoconazole, oxiconazole, and fenticonazole.

In one preferred embodiment, the azole fuingicide for human application is itraconazole and the invention provides a hydrophilic inclusion complex consisting essentially of nanosized particles of amorphous itraconazole wrapped in polyacrylic acid or in an acrylic acid-butyl acrylate copolymer.

The active compound may also be an azole that acts as nonsteroidal antiestrogens and can be used in the treatment of estrogen-responsive breast tumors in postmenopausal women, including, but not limited to letrozole, anastrozole, vorozole, and fadrozole, or an azole fuingicide useful in the agriculture including, but not limited to, the triazoles bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, and the imidazoles imazalil, prochloraz, and triflumizole. In still another embodiment, the azole compound is a nonfungicidal azole for use in the agriculture such as the triazoles azocyclotin used as an acaricide, paclobutrazole as a growth regulator, carfentrazone as a herbicide, and isazophos as an insecticide, and the imidazole metazachlor used as herbicide.

In another embodiment, the amorphous active compound is donepezil hydrochloride and the invention relates to a hydrophilic inclusion complex consisting essentially of nanosized particles of amorphous donepezil hydrochloride wrapped in a polysaccharide selected from the group consisting of natural or modified starch or alginate. The modified starch may be hydrolyzed potato starch or sodium starch glycolate.

The nanoparticles of the present invention comprise the active compound or core wrapped within a water-soluble amphiphilic polymer. As described in the parent U.S. application Ser. No. 10/256,023 and Ser. No. 09/966,847, hereby incorporated by reference in their entirety, a variety of different polymers can be used for any of the selected active compounds. The polymer used in the formation of the nano-soluparticles are selected according to an algorithm that takes into account various physical properties of the active compounds and the polymer or polymers, as well as their future interaction in the resulting complex. The algorithm is utilized in this manner to select the optimal polymer(s) and takes into consideration the following properties of the polymer itself in selecting a polymer for the active molecule/polymer interaction in the formation of the complex: molecular weight, basic polymer chain length, the length of the kinetic unit, the solubility of the polymer in water, the overall degree of solubility, the degree of polymer flexibility, the hydrophilic-lipophilic balance (HLB), and the polarity of the hydrophilic groups of the polymer. The main properties of the polymer include its HLB, the length and the flexibility of its polymer chain, and also the state of polarity of the hydrophilic groups.

Thus, one important parameter in the choice of the polymer or polymers is the HLB, i.e., the measure of the molecular balance of the hydrophilic and lipophilic portions of the compound. Within the HLB International Scale of 0-20, lipophilic molecules have a HLB of less than 6, and hydrophilic molecules have a HLB of more than 6. Thus, according to the present invention, the HLB of the polymer is selected in such a way that, after combining to it the active compound, the total resulting HLB value of the complex will be greater than 8, rendering the complex water-soluble.

In another aspect, the present invention provides a hydrophilic dispersion comprising nanoparticles of inclusion complexes as defined above. Thus, the present invention provides a hydrophilic dispersion of water-soluble and stable nanoparticles of inclusion complexes consisting essentially of nanosized particles of an active compound in amorphous form and an amphiphilic which wraps said active compound such that non-valent bonds are formed between said active compound and said amphiphilic polymer in said inclusion complex.

The dispersions of the invention are stable. Stability of the nanoparticles and of the inclusion complexes has more than one meaning. The nanoparticles should be stable as part of a nanocomplex over time, while remaining in the dispersion media. The nanodispersions are stable over time without separation of phases. Furthermore, the amorphous state is also retained over time.

It is worth noting that in the process used in the present invention, the components of the system do not result in micelles nor do they form classical dispersion systems. The technology of the present invention causes the following:
(i) after dispersion of the active macromolecule to nanosized particles and fixation by the polymer to form an inclusion complex, enhanced solubility in physiological fluids, in vivo improved absorption, and improved biological activity, as well as transmission to a stable amorphous, state, are achieved; and
(ii) the otherwise crystalline biologically-active compound becomes amorphous, and thus exhibits improved biological activity.

In most preferred embodiments of the present invention, not less than 80% of the nanoparticles in the nanodispersion are within the size range, when the size deviation is not greater than 20%, and the particle size is within the nano range, namely less than 1000 nm, more preferably 100 nm or less.

In an advantageous and preferred embodiment of the invention, the polysaccharide molecule “wraps” the active compound via non-valent interactions. The non-valent bonds or interactions such as electrostatic forces, van der Waals forces, and hydrogen bonds formed between the polysaccharide and the active compound in the inclusion complex fixate the active compound within the polymer, thus reducing its molecular mobility. The formation of any valent bonds could change the characteristics or properties of the active compound. The formation of non-valent bonds preserves the structure and properties of the active compound, which is particularly important when the active compound is a pharmaceutical.

The hydrophilic dispersions of the present invention can be prepared by the process described in the above-mentioned parent U.S. application Ser. No. 10/952,380, Ser. No. 10/256,023, and Ser. No. 09/966,847, incorporated herewith by reference in their entirety.

The aqueous nanodispersions of the invention can be lyophilized and then mixed with pharmaceutically, cosmetically or agriculturally acceptable carriers to provide stable pharmaceutical, cosmetic or pesticidal compositions, respectively.

The invention will now be illustrated by the following non-limiting examples.

### EXAMPLES

### General Methods

(i). General Procedure for Preparation of Dispersions of Nanoparticles of Inclusion Complexes of Amorphous Active Compounds (Solumerization of Active Compounds)

For the preparation of the hydrophilic dispersion comprising the nanoparticles of the invention, the following general procedure is carried out:
(i) preparation of a molecular solution of the amphiphilic polymer in water;
(ii) preparation of a molecular solution of the active compound in an organic solvent;
(iii) dripping the cold solution (ii) of the active compound into the polymer solution (i) heated at a temperature 5-10° C. above the boiling point of the organic solvent of (ii), under constant mixing; and
(iv) evaporation of the organic solvent, thus obtaining the desired hydrophilic dispersion comprising nanoparticles of the inclusion complexes of the active compound in amorphous form wrapped in the amphiphilic polymer.
   (ii) X-Ray Diffraction Analysis

X-ray diffraction gives very distinct patterns for crystalline and amorphous materials. The diffracting X-rays interact with the variation of electron density inside the sample. For crystalline material, the periodic repeating electron density will give rise to well defined diffraction peaks whose widths are determined by the crystalline “quality”. Highly crystalline material will give rise to sharp peaks (high frequency) whose widths are limited by the instrumental resolution, while non-crystalline material will give rise to broader and more diffuse diffraction peaks (low frequency). Amorphous materials may come in different forms depending on their formation. If the formation is a glassy amorphous phase, then the diffraction signal is the radial distribution of nearest neighbor molecular interactions. On the other hand, if the amorphous phase is derived from the crystalline phase, then usually it corresponds to para-crystalline material. Para-crystalline material will either generate extremely broad peaks corresponding to the crystalline peaks, or it will diffract intensity corresponding to the diffraction from a single unit cell (Unit Cell Structure Factor). Whether glassy or para-crystalline, the amorphous diffraction is usually a broad very low frequency halo with occasional harmony. The crystalline component is more like para-crystalline material in nature with very broad peaks.

The following X-ray method and equipment were used: X-ray diffraction patterns were collected with CuKa radiation using a Scintag theta-theta powder diffractometer equipped with a liquid nitrogen-cooled solid-state Ge detector.

(iii) Differential Scanning Calorimetry (DSC) Analysis

DSC was done with a TA Instruments 2010 module and a 2100 System Controller to study the crystallinity of complexes. Prior to analysis, the samples are sealed in alodined aluminum DSC pans. The tests are done at a scan rate of 10 degrees/minute, from −50 to 200° C.

(iv). Measurement of Particle Size By Light Scattering Analysis and Electron Microscopy

The size of nanoparticles of inclusion complexes was analyzed using two methods: light scattering and cryo-transmission electron microscopy (TEM). Light scattering measurements of the nanoparticles size were performed using ALV-Particle Sizer (ALV-Laser GmbH, Langen, Germany), which has a resolution of 3-3000 nm. ALV is a dynamic light scattering technique used to estimate the mean particle size. Experiments were conducted with a laser-powered Noninvasive Back Scattering High Performance Particle Sizer (ALV-NIBS/HPPS). A 1:10 dilution of the samples was found necessary for sample analysis by this method.

### Example 1

### Donepezil Hydrochloride (HD) Dispersions (DH Solumers)

Crystalline donepezil hydrochloride (DH) powder was used to produce the amorphous DH hydrophilic dispersions. These dispersions were prepared by the method described in General Methods (i) above with an aqueous solution comprising 4% hydrolyzed potato starch (HPS; AVEBE Group, The Netherlands), with the difference that DH was dissolved in two different solvents: in 78.5 ml dichloromethane (DClM; chemically pure; Frutarom, Israel) and in parallel with 350 ml methyl acetate (MA; chemically pure; Merck), and gradually added to the polymer aqueous solution to achieve a final concentration of 1%, after evaporation of the solvent. Methyl acetate was added to prevent bubbling during the process.

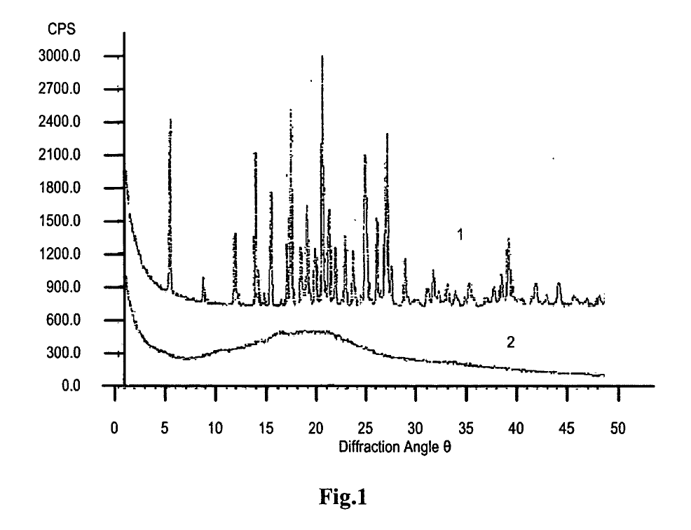

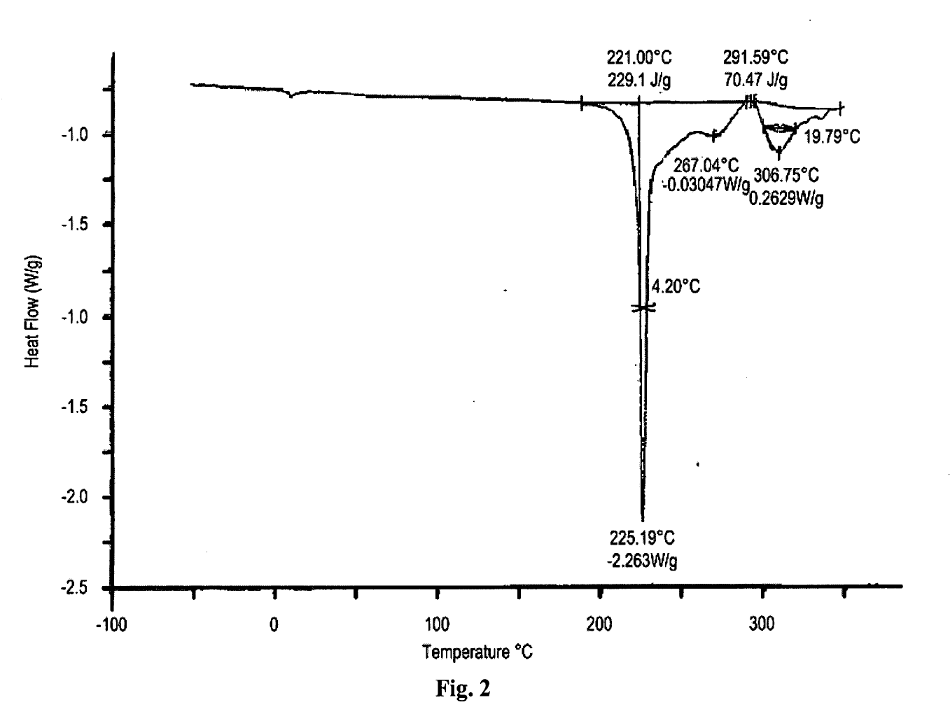

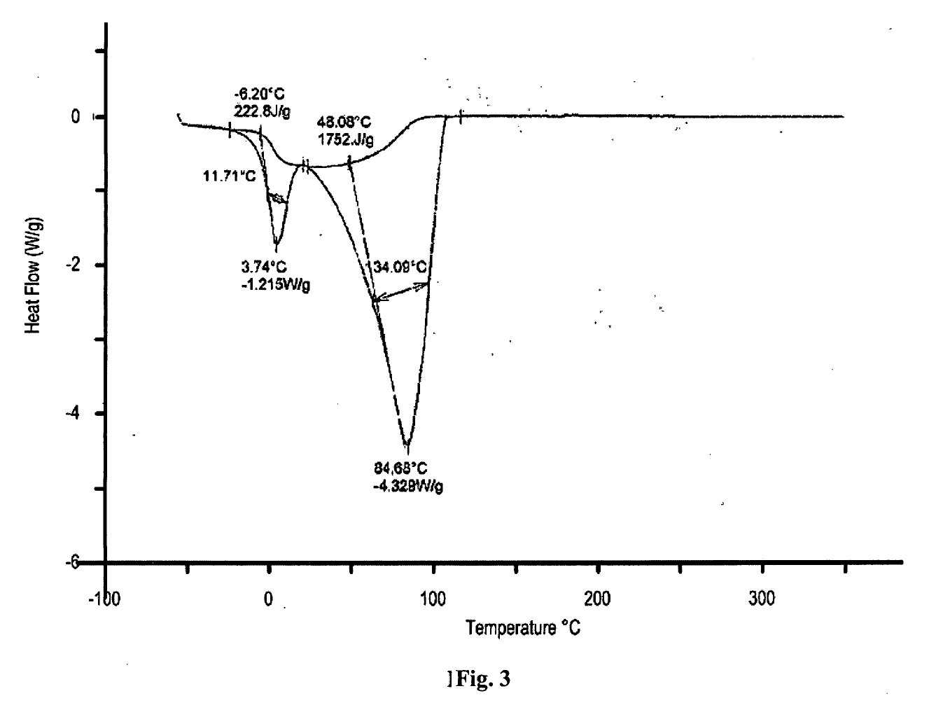

FIG. 1 demonstrates that DH powder is crystalline (curve 1) and the DH-HPS Solumer is amorphous (curve 2), and FIGS. 2 and 3 further support this observation: in FIG. 2 it can be seen that DH crystals melt at the characteristic melting point (225.19° C.), while FIG. 3 shows that DH in the DH-HPS Solumer does not melt at the characteristic point (it melts at 84.68 ° C.), further supporting the X-ray data.

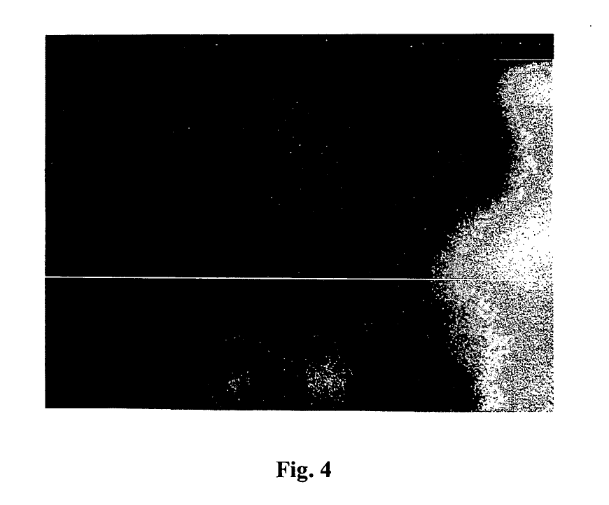

Similarly, other dispersions comprising up to 6% DH were prepared such that the X-ray and DSC analyses indicated that DH is amorphous. In some of these dispersions, the DH-polymer complexes were nanoparticles (FIG. 4).

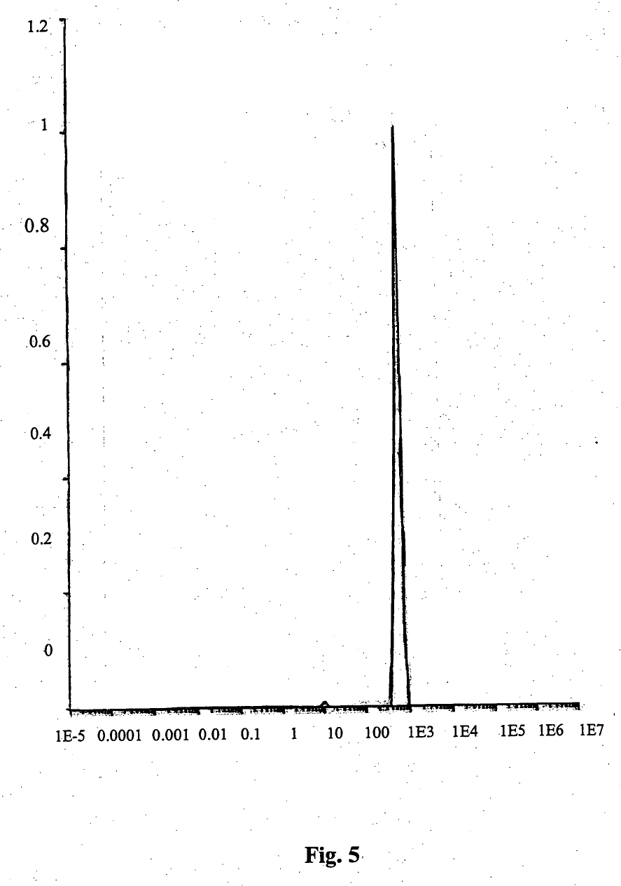

Table 1 below shows the properties of various such donepezil hydrochloride hydrophilic inclusion complexes. FIG. 5 illustrates the size distribution of nano-particles comprising donepezil hydrochloride hydrophilic inclusion complexes within modified corn starch (#LG-7-51) having a size of approximately 600 nm. However, though amorphous donepezil hydrochloride was apparent, particles of some of these dispersions had diameters significantly greater than 1 micron. Thus, donepezil hydrochloride in these dispersions was amorphous regardless of the particle size.

TABLE 1
Properties of donepezil HCl (DH) hydrophilic inclusion complexes

| | | | | HPLC | | | |
|---|---|---|---|---|---|---|---|
| | Polymer | DH | | After | ALV | | |
| Exp. | (name/%) | (%) | pH | dry % | nm | X-Ray | DSC |
| IC-130 | 2% Alginate | 2 | 5.2 | 97 | ND | Amorphous | Amorphous |
| | (Kelton) LV | | | | | | |
| LG-7-38 | 2% Na Starch | 1 | 5.5 | 80 | ND | ND | Amorphous |
| | Glycolate | | | | | | |
| | (Explotab) | | | | | | |
| LG-7-44 | 1% Alginate | 1 | 5 | 103 | ND | ND | Amorphous |
| | (Kelton) LV | | | | | | |
| LG-7-51 | 2% Corn Starch | 1 | 5 | 104 | 600 | Amorphous | ND |
| | pregelatinized, | | | | | | |
| | modified | | | | | | |
| | (PureCote ™) | | | | | | |
| | B-793 | | | | | | |

HPLC = High Performance Liquid Chromatography assay;
ND = not done.

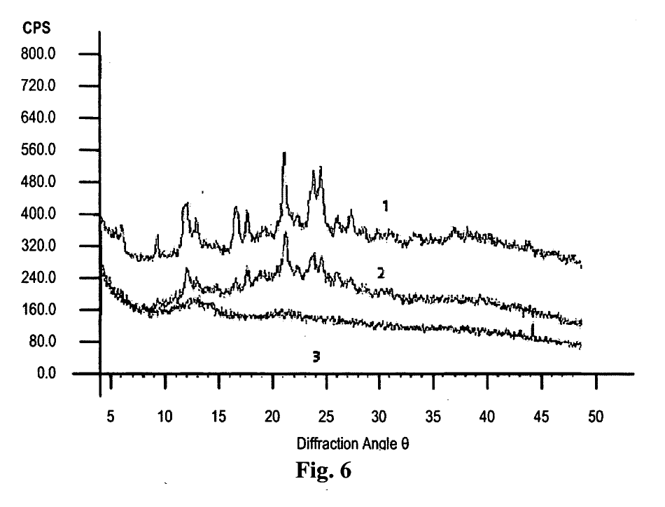

Selection of both polymer and process conditions were found to have an impact on the physical characteristics of the obtained donepezil hydrochloride inclusion complexes. Besides HPS, sodium alginate (Kelco), modified corn starch B-793 (Instant Pure-Cotee, Grain Processing Corp., Muscatine, Iowa), and sodium starch glycolate (the sodium salt of a carboxymethyl ether of starch) were among the polymers also found usefuil for preparing dispersions with non-crystalline donepezil hydrochloride (as exemplified in FIG. 6 for DH-alginate Solumer).

Additionally, the relative amounts of dichloromethane and methyl acetate impacted the physical characteristics of the obtained donepezil hydrochloride complexes. FIG. 6 shows that addition of methyl acetate and dichloromethane in a ratio of 1/10 (v/v) or use of dichloromethane alone, yielded DH-alginate dispersions having DH in the disordered crystalline state. Furthermore, completely amorphous donepezil hydrochloride dispersions were obtained when the amount of methyl acetate added was at least that of dichloromethane (as shown in FIG. 1).

Since the non-crystalline state has been previously observed (Yonemochi E. et al., 1999 Eur. J. Pharm. Sci. 7:331-338) to be temporary (<1 week), the crystallinity of these samples was monitored as a function of time. The dispersions described above were analyzed by X-ray diffraction, during extended storage periods at room temperature. So far, it has been observed that the amorphous state is retained for periods of at least nine months.

### Example 2

### Itraconazole Solumer Dispersions

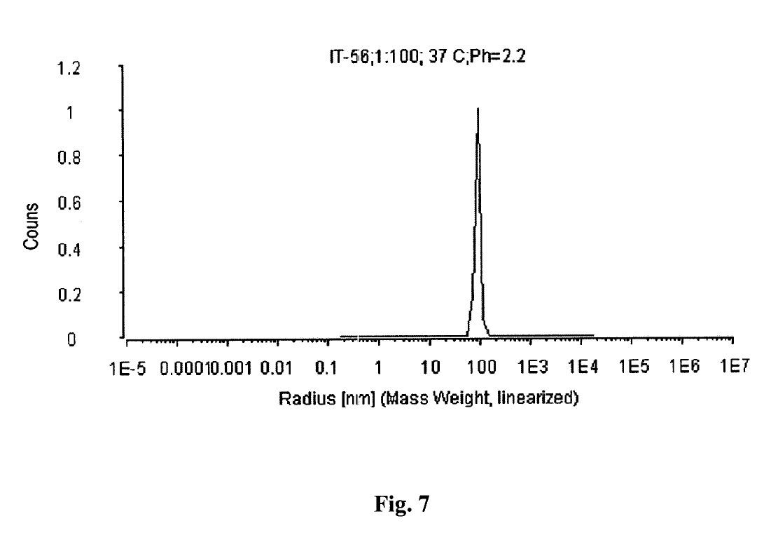

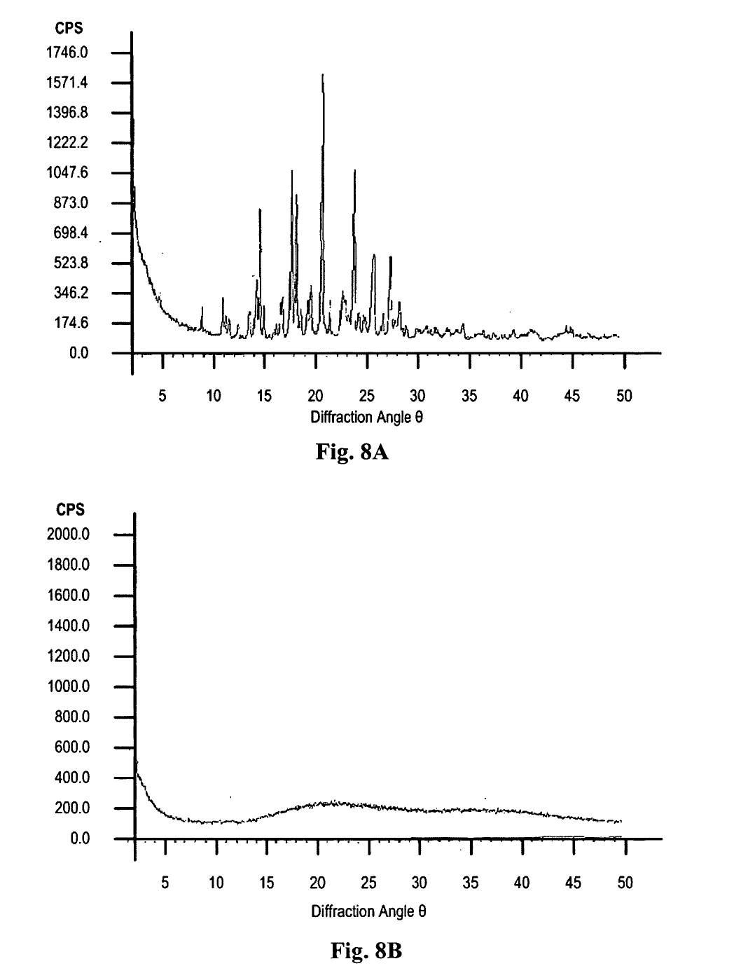

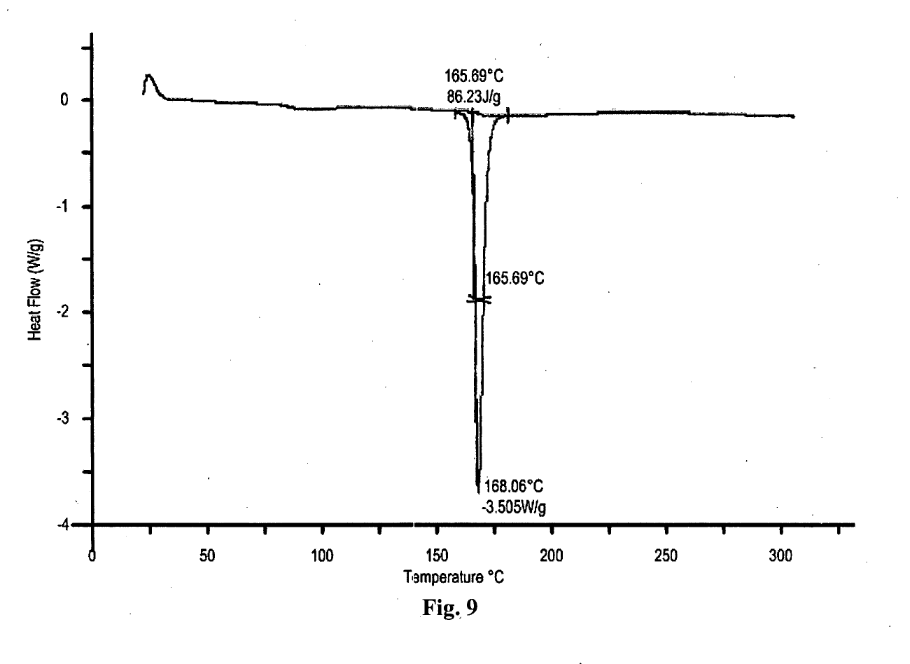

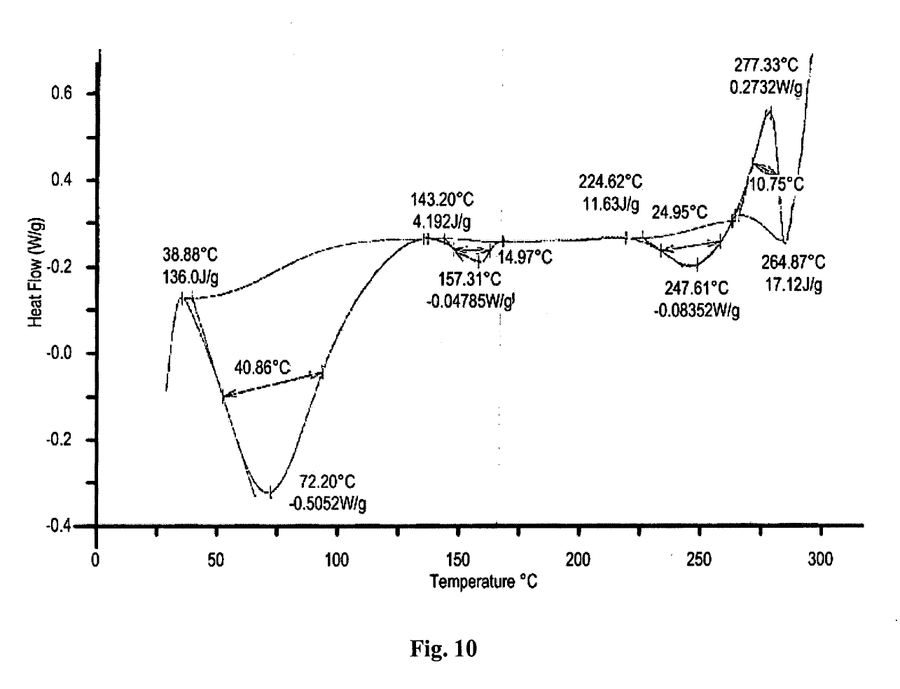

Dispersions of nanoparticles, in water, were prepared from crystalline itraconazole using acrylate copolymers. These dispersions were prepared by the method described in General Methods (i) with an aqueous solution comprising 30% copolymer of acrylic acid (Merck) and butyl acrylate (Merck). Itraconazole (IT) was gradually added, in 250 ml methyl acetate (MA), to achieve a final concentration of 1.5%, after evaporation of the solvent. As demonstrated by light scattering (FIG. 7), the particles in dispersions prepared by this method, have a diameter of approximately 100 nm, and the size distribution is very narrow. FIG. 8 demonstrates that IT powder is crystalline, while FIG. 8 demonstrates that the IT Solumer dispersion is amorphous. FIGS. 9 and 10 further support this observation. In FIGS. 9 and 10, it can be seen that, while IT crystals melt at the characteristic melting point (FIG. 9), IT, in IT-copolymer dispersion (FIG. 10), does not melt at the characteristic point, further supporting the X-ray data.

Table 2 shows the properties of various itraconazole hydrophilic inclusion complexes in copolymer acrylic acid-butyl acrylate.

TABLE 2
Properties of itraconazole hydrophilic inclusion complexes

| | | Drug | HPLC | Particle |
|---|---|---|---|---|
| | | (mg/ | % of | Size |
| Exp | Polymer (name/%) | ml) | Initial | nm |
| IT-50 | 30% Co-polymer | 10 | 74.2 | 70-80 |
| | (acrylic acid 26.25% and butyl | | | |
| | acrylate 3.75%) | | | |
| IT-51 | 43.75% Co-polymer | 10 | 70.8 | 70-80 |
| | (acrylic acid 38.25% and butyl | | | |
| | acrylate 5.5%) | | | |
| IT-52 | 33.33% Co-polymer | 10 | 85.5 | 68-109 |
| | (acrylic acid 29.33% and butyl | | | |
| | acrylate 4%) | | | |
| IT-OS-38-17 | 30% Co-polymer | 12 | 95.5 | 67 |
| | (acrylic acid:butyl acrylate | | | |
| | 24:1) | | | |
| IT-56 | 33.3% polymer (acrylic acid) | 10 | 91.9 | 85 |

HPLC = High Performance Liquid Chromatography assay

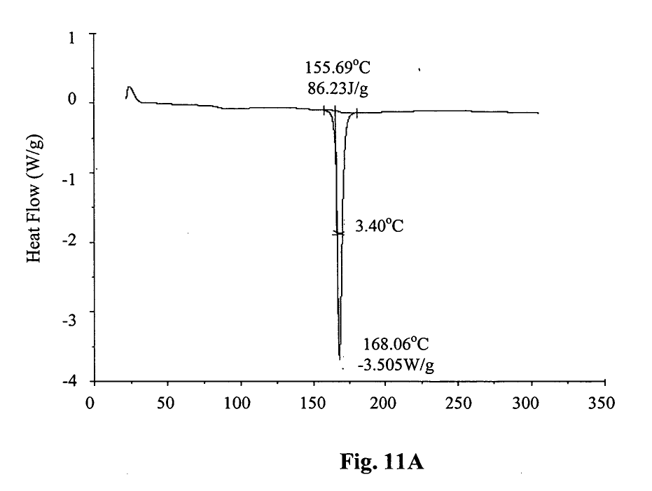

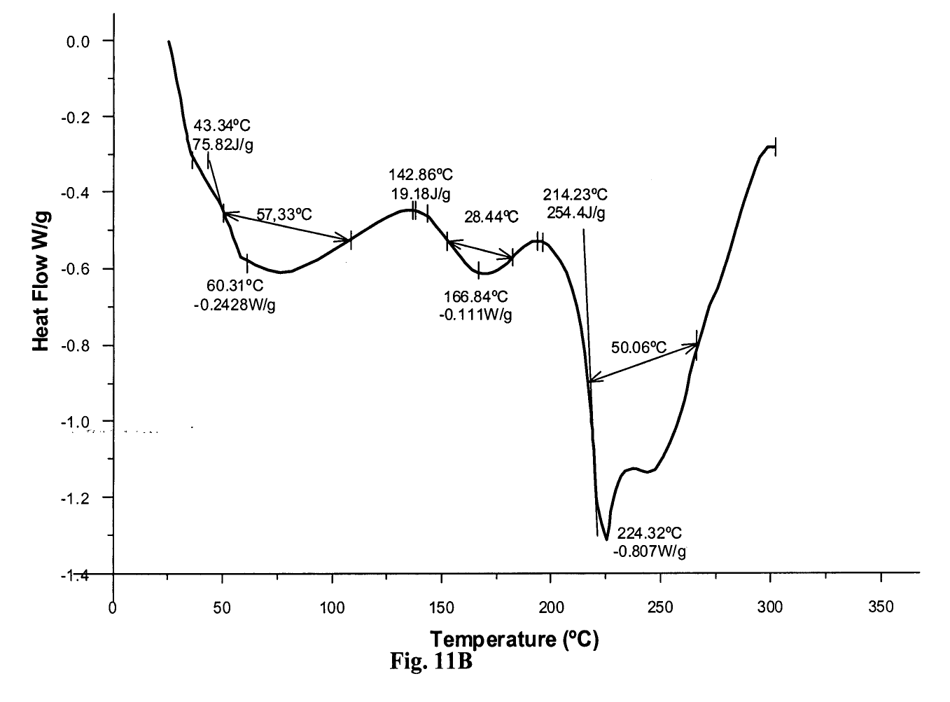

FIGS. 11A-11B provide illustrations of itraconazole crystals and the itraconazole complexes in polyacrylic acid prepared in experiment IT-56 (see Table 2), respectively. While itraconazole crystals melt at the characteristic melting point, itraconazole complexes do not melt at the characteristic point

### Example 3

### Azithromycin Solumer Dispersions

Crystalline azithromycin (AZI) powder was used to produce amorphous AZI in dispersions in water. These dispersions were prepared by the method described in General Methods (i) with a solution comprising 4% hydrolyzed potato starch (HPS). AZI in 500 ml methyl acetate (MA) was gradually added to the HPS solution, to achieve a final concentration of 1%, after evaporation of the solvent. MA was added to prevent bubbling during the process.

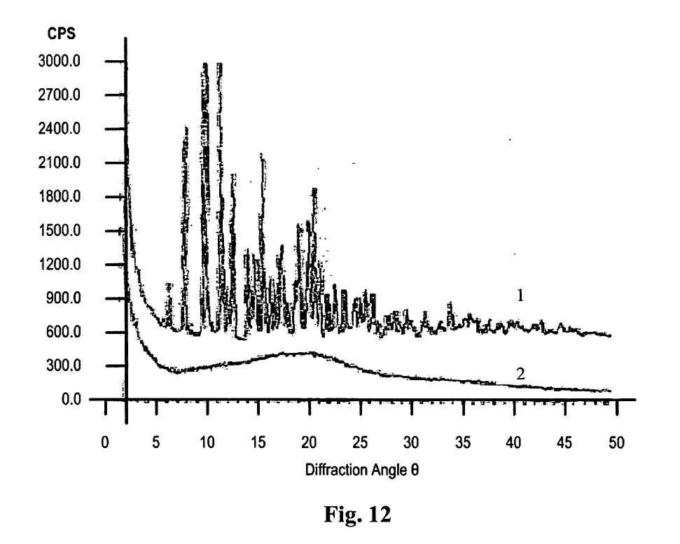

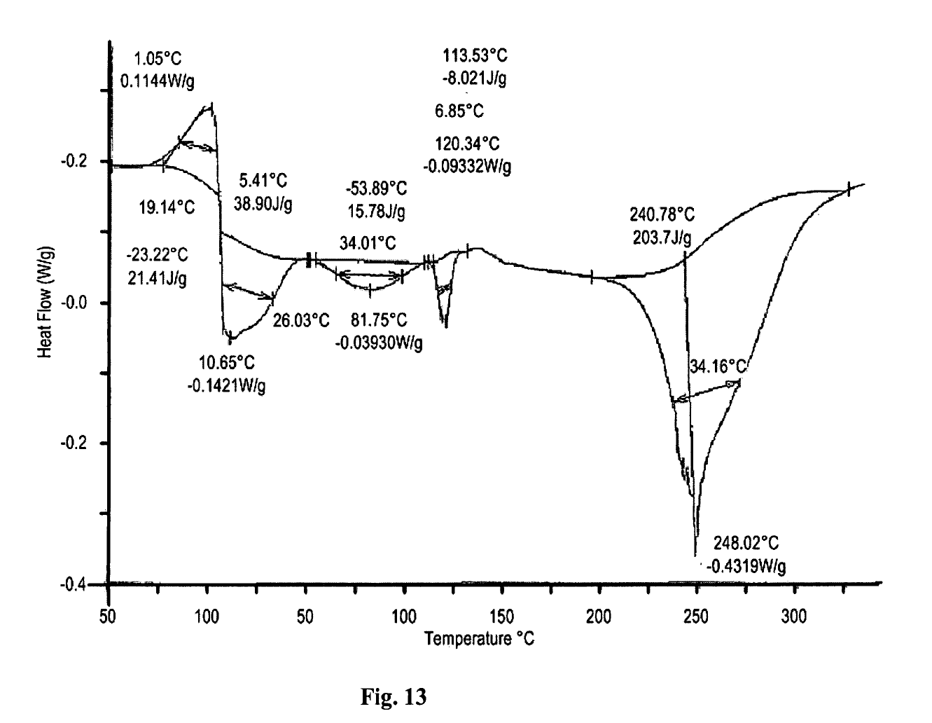

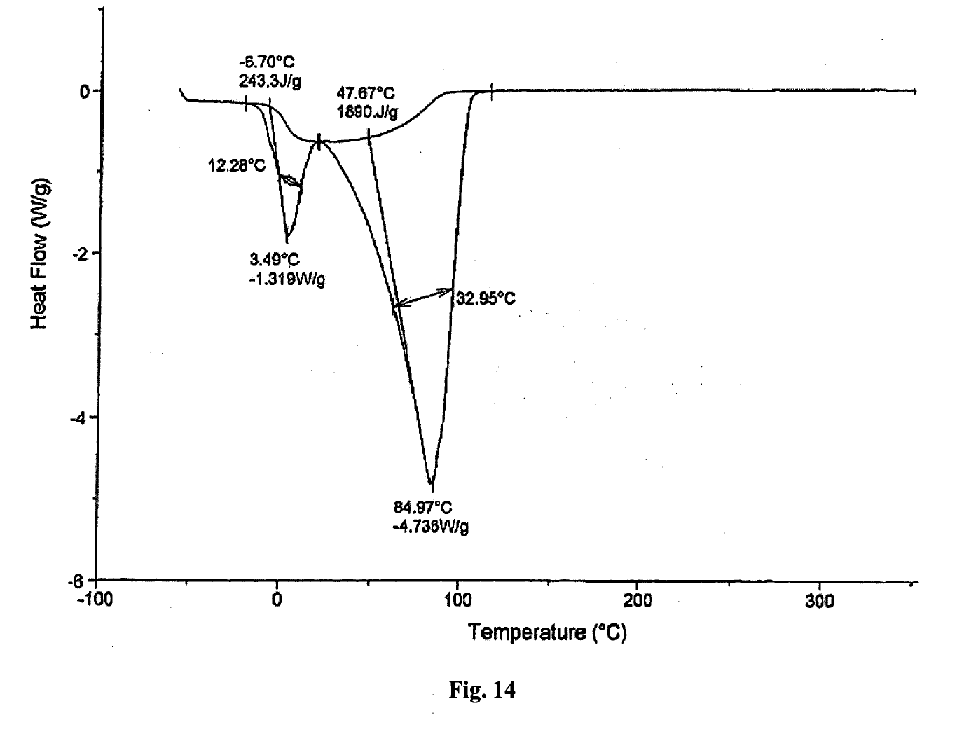

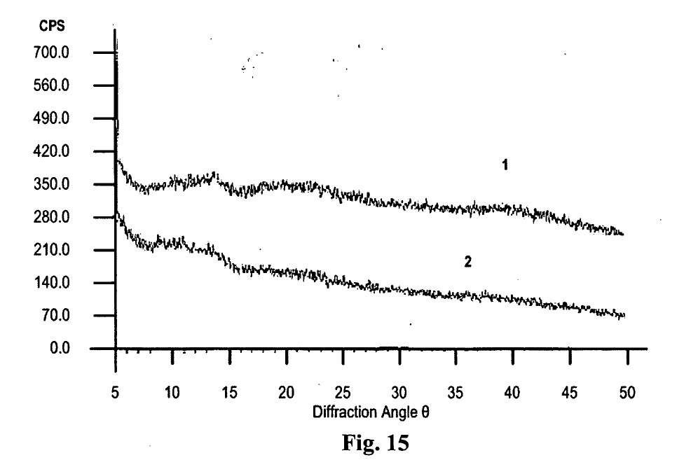

FIG. 12 demonstrates that AZI powder is crystalline, and AZI, in the solumer dispersion, is amorphous. FIGS. 13 and 14 further support this observation. In FIGS. 13 and 14, it can be seen that, while AZI crystals melt at the characteristic melting point (FIG. 13), AZI, in AZI-HPS dispersions, does not melt at the characteristic point (FIG. 14), fuirther supporting the X-ray data. Similarly, other dispersions prepared with either chitosan (Kraeber GmbH) or alginate, were prepared such that the X-ray analyses indicated that AZI is amorphous. For example, FIG. 15 shows that dispersions prepared with alginate are still amorphous for at least 6 months. In some of these dispersions, the AZI1-polymer complexes were nanoparticles (data not shown).

Table 3 below shows the properties of various inclusion complexes of azithromycin with 1% chitosan and 2% alginate, prepared according to the method described in General Methods (i), in which azithromycin was dissolved in methyl acetate or dichloromethane. Shown in Table 3 are complex designation (Exp., first column), polymer name and concentration (%), drug concentration, pH, and physicochemical analysis of the various complexes nanoparticles including ALV-size and size distribution (nm) and HPLC (concentration and thus solubility).

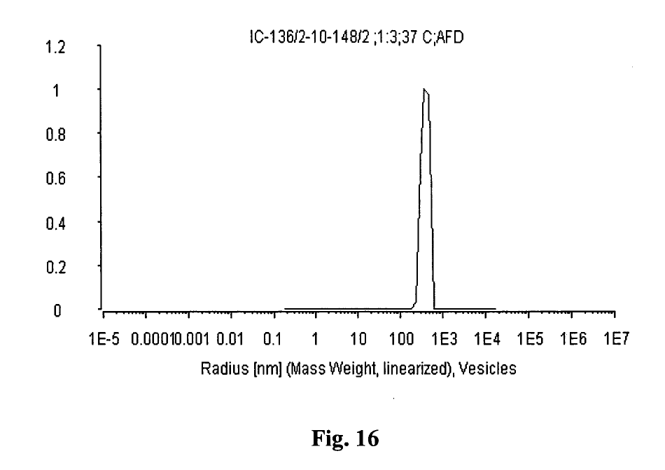

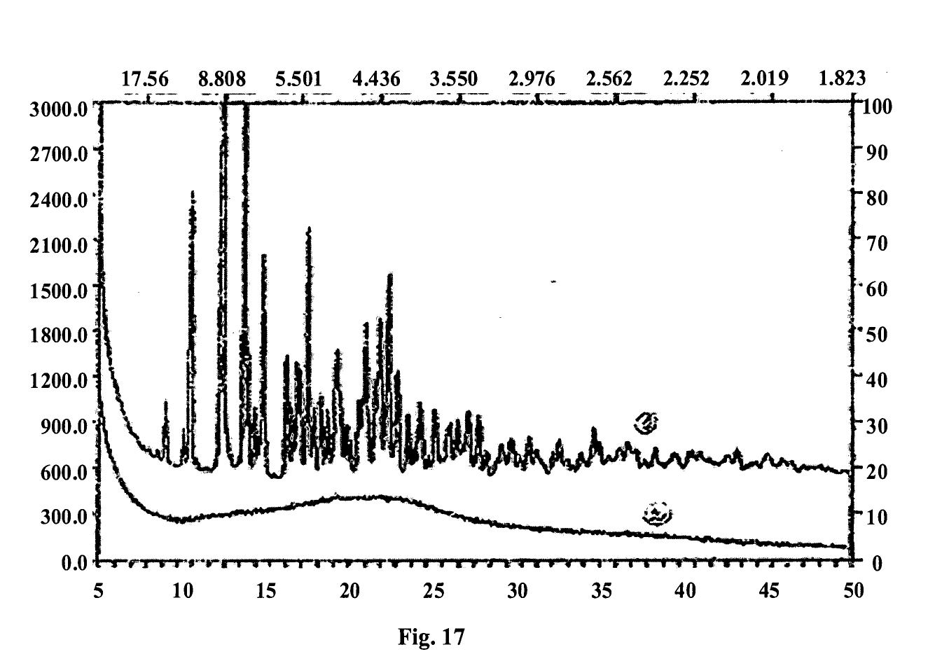

FIG. 16 illustrates the size distribution of nanoparticles of the azithromycin hydrophilic inclusion complex within 1% chitosan (# 10-148/2 in Table 3) having a size of approximately 362 nm. Furthermore, azithromycin in these particles was found to be amorphous, and as shown in the lower curve of FIG. 17, the amorphousity was found to be stable for at least ten months.

TABLE 3
Properties of Azithromycin Hydrophilic Inclusion Complexes

| | | | HPLC | Particle |
|---|---|---|---|---|
| | Polymer | Drug | % of | Size |
| Exp. | (name/%) | (mg/ml) | Initial | nm |
| AZ-IC-131/1-IZ-10-145 | 2% Alginate | 20 | 82.6 | 1600 |
| | (Kelton LV) | | | |
| AZ-IC-134/1-IZ-10-147 | 2% Alginate | 10 | 98.06 | 1060 |
| | (Kelton LV) | | | |
| AZ-IC 136/2-IZ-10-148 | 1% Chitosan | 10 | 97.16 | 510 |
| AZ-IC 136/3-IZ-28-1 | 1% Chitosan | 10 | 95.36 | 752 |
| AZ-IC 136/2-10-148/2 | 1% Chitosan | 10 | 97 | 362 |

HPLC = High Performance Liquid Chromatography assay

The complexes with 1% chitosan (Sigma C3646) and with 2% sodium alginate (Kelton L V, from Kelco Co., San Diego, Calif., USA) were found to be amorphous.

### Example 4

### Clarithromycin Solumer Dispersions

Dispersions of clarithromycin hydrophilic inclusion complexes were prepared according to the method described in General Methods (i), in which clarithromycin was dissolved in methyl acetate or dichloromethane and the polymers were hydrolyzed potato starch, alginate, or chitosan.

Table 4 below shows the properties of various such complexes. Shown in Table 4 are complex designation (Exp., first column), polymer name and concentration (%), drug concentration, pH, and physico-chemical analysis of the various complexes nanoparticles including ALV-size and size distribution (nm), HPLC (concentration and thus solubility) and powder X-ray analyses for the determination of crystalline phase.

TABLE 4
Properties of Clarithromycin Hydrophilic Inclusion Complexes

| | | | | HPLC | | Size | |
|---|---|---|---|---|---|---|---|
| | Polymer | Drug | | Quantity | % of | Distribution | |
| Exp. | (name/%) | (mg/ml) | pH | (ml) | Initial | nm | X-Ray |
| IC-98 | Hydrolyzed | 10 | 5 | 5 | 93.9 | ND | Amorphous |
| (75) | potato starch | | | | | | |
| | 4% dil to 2% | | | | | | |
| IC-133 | 2% Alginate | 10 | 5.5 | 20 | 44.3 | 530 | Amorphous |
| | Kelton LV | | | | | | |
| IC-135 | 1% Chitosan | 10 | 4-6 | 5 | 84.5 | 165 | Amorphous |
| | Fluka | | | | | | |
| | 50494 | | | | | | |

Dil = diluted;
LV = low viscosity;
HPLC = High Performance Liquid Chromatography;
ND = not done

As shown in Table 4, nanoparticles (size below 1000 nm) of amorphous clarithromycin could be prepared using polymers such as hydrolyzed potato starch, alginate, and chitosan.

1. A hydrophilic inclusion complex consisting essentially of nanosized particles of an active compound in amorphous form and an amphiphilic polymer which wraps the active compound such that non-valent bonds are formed between the active compound and the amphiphilic polymer in said inclusion complex.

2. The hydrophilic inclusion complex according to claim 1, wherein said amphiphilic polymer is selected from the group consisting of natural polysaccharides, modified polysaccharides, polyacrylic acid and copolymers thereof, polymethacrylic acid and copolymers thereof, polyacrylamide and copolymers thereof, polymethacrylamide and copolymers thereof, polyethylene imine, polyethylene oxide, polyvinyl alcohol, polyisoprene, polybutadiene and gelatin.

3. The hydrophilic inclusion complex according to claim 2, wherein said amphiphilic polymer is a polysaccharide selected from the group consisting of natural or modified starch, chitosan and alginate.

4. The hydrophilic inclusion complex according to claim 1, wherein said active compound in amorphous form is selected from the group consisting of pharmaceutical compounds, food additives, cosmetics, pesticides and pet foods.

5. The hydrophilic inclusion complex according to claim 4, wherein said active compound in amorphous form is a pharmaceutical compound.

6. The hydrophilic inclusion complex according to claim 5, wherein said active compound in amorphous form is a macrolide antibiotic selected from the group consisting of erythromycin, clarithromycin and azithromycin.

7. The hydrophilic inclusion complex according to claim 6, wherein said macrolide antibiotic is azithromycin.

8. The hydrophilic inclusion complex according to claim 7, consisting essentially of nanosized particles of amorphous azithromycin wrapped in a polysaccharide selected from the group consisting of natural or modified starch, chitosan or alginate.

9. The hydrophilic inclusion complex according to claim 8, wherein said polysaccharide is hydrolyzed potato starch (HPS).

10. The hydrophilic inclusion complex according to claim 6, wherein said macrolide antibiotic is clarithromycin.

11. The hydrophilic inclusion complex according to claim 10, consisting essentially of nanosized particles of amorphous clarithromycin wrapped in a polysaccharide selected from the group consisting of starch, chitosan or alginate.

12. The hydrophilic inclusion complex according to claim 11, wherein said polysaccharide is hydrolyzed potato starch (HPS).

13. The hydrophilic inclusion complex according to claim 4, wherein said amorphous active compound is an azole compound.

14. The hydrophilic inclusion complex according to claim 13, wherein the azole compound is an imidazole or triazole compound for human or veterinary application or for use in the agriculture.

15. The hydrophilic inclusion complex according to claim 14, wherein the azole compound is an azole fungicide for human application selected from the group consisting of terconazole, itraconazole, fluconazole, clotrimazole, miconazole, econazole, ketoconazole, tioconazole, isoconazole, oxiconazole, and fenticonazole.

16. The hydrophilic inclusion complex according to claim 15, wherein the azole fuingicide is itraconazole.

17. The hydrophilic inclusion complex according to claim 16, consisting essentially of nanosized particles of amorphous itraconazole wrapped in polyacrylic acid or in an acrylic acid-butyl acrylate copolymer.

18. The hydrophilic inclusion complex according to claim 5, wherein said amorphous active compound is donepezil hydrochloride.

19. The hydrophilic inclusion complex according to claim 18, consisting essentially of nanosized particles of amorphous donepezil hydrochloride wrapped in a polysaccharide selected from the group consisting of natural or modified starch or alginate.

20. The hydrophilic inclusion complex according to claim 19, wherein the donepezil hydrochloride is wrapped in hydrolyzed potato starch or sodium starch glycolate.

21. A hydrophilic dispersion comprising nanoparticles of inclusion complexes consisting essentially of nanosized particles of an active compound in amorphous form and an amphiphilic polymer which wraps the active compound such that non-valent bonds are formed between the active compound and the amphiphilic polymer in said inclusion complex.

22. The hydrophilic dispersion according to claim 21, wherein said amphiphilic polymer is selected from the group consisting of natural polysaccharides, modified polysaccharides, polyacrylic acid and copolymers thereof, polymethacrylic acid and copolymers thereof, polyacrylamide and copolymers thereof, polymethacrylamide and copolymers thereof, polyethylene imine, polyethylene oxide, polyvinyl alcohol, polyisoprene, polybutadiene and gelatin.

23. The hydrophilic dispersion according to claim 22, wherein said amphiphilic polymer is a polysaccharide selected from the group consisting of natural or modified starch, chitosan and alginate.

24. The hydrophilic dispersion according to claim 21, wherein said active compound in amorphous form is selected from the group consisting of pharmaceutical compounds, food additives, cosmetics, pesticides and pet foods.

25. The hydrophilic dispersion according to claim 24, wherein said active compound in amorphous form is a pharmaceutical compound.

26. The hydrophilic dispersion according to claim 25, wherein said active compound in amorphous form is a macrolide antibiotic selected from the group consisting of erythromycin, clarithromycin and azithromycin.

27. The hydrophilic dispersion according to claim 26, wherein said macrolide antibiotic is azithromycin.

28. The hydrophilic dispersion according to claim 27, consisting essentially of nanosized particles of amorphous azithromycin wrapped in a polysaccharide selected from the group consisting of natural or modified starch, chitosan or alginate.

29. The hydrophilic dispersion according to claim 28, wherein said polysaccharide is hydrolyzed potato starch (HPS).

30. The hydrophilic dispersion according to claim 26, wherein said macrolide antibiotic is clarithromycin.

31. The hydrophilic dispersion according to claim 30, consisting essentially of nanosized particles of amorphous clarithromycin wrapped in a polysaccharide selected from the group consisting of natural or modified starch, chitosan or alginate.

32. The hydrophilic dispersion according to claim 31, wherein said polysaccharide is hydrolyzed potato starch (HPS).

33. The hydrophilic dispersion according to claim 24, wherein said amorphous active compound is an azole compound.

34. The hydrophilic dispersion according to claim 33, wherein the azole compound is an imidazole or triazole compound for human or veterinary application or for use in the agriculture.

35. The hydrophilic dispersion according to claim 34, wherein the azole compound is an azole fungicide for human application selected from the group consisting of terconazole, itraconazole, fluconazole, clotrimazole, miconazole, econazole, ketoconazole, tioconazole, isoconazole, oxiconazole, and fenticonazole.

36. The hydrophilic dispersion according to claim 35, wherein the azole fungicide is itraconazole.

37. The hydrophilic dispersion according to claim 36, consisting essentially of nanosized particles of amorphous itraconazole wrapped in polyacrylic acid or in an acrylic acid-butyl acrylate copolymer.

38. The hydrophilic dispersion according to claim 25, wherein said amorphous active compound is donepezil hydrochloride.

39. The hydrophilic dispersion according to claim 38, consisting essentially of nanosized particles of amorphous donepezil hydrochloride wrapped in a polysaccharide selected from the group consisting of natural or modified starch or alginate.

40. The hydrophilic dispersion according to claim 39, wherein the donepezil hydrochloride is wrapped in hydrolyzed potato starch or sodium starch glycolate.

41. A stable composition comprising a hydrophilic dispersion according to claim 21 and a carrier.

42. A stable pharmaceutical composition according to claim 41 comprising said hydrophilic dispersion and a pharmaceutically acceptable carrier.

43. A stable pharmaceutical composition according to claim 42, comprising a hydrophilic dispersion of nanosized particles of amorphous donepezil hydrochloride wrapped in a polysaccharide selected from the group consisting of natural or modified starch or alginate, and a pharmaceutically acceptable carrier.

44. A stable pharmaceutical composition according to claim 42 comprising a hydrophilic dispersion of nanosized particles of amorphous itraconazole wrapped in polyacrylic acid or in an acrylic acid-butyl acrylate copolymer, and a pharmaceutically acceptable carrier.

45. A stable pharmaceutical composition according to claim 42, comprising a hydrophilic dispersion of nanosized particles of amorphous azithromycin or clarithromycin wrapped in a polysaccharide selected from the group consisting of natural or modified starch, chitosan or alginate, and a pharmaceutically acceptable carrier.
